(19) United States
(12) Patent Application Publication
(10) Pub. No.: US 2020/0352532 A1
(43) Pub. Date: Nov. 12, 2020

(54) RADIOGRAPHY CONTROL DEVICE AND RADIOGRAPHY SYSTEM

(71) Applicant: Konica Minolta Inc., Tokyo (JP)

(72) Inventor: TAKAHISA UEHARA, Yokohama-shi (JP)

(21) Appl. No.: 16/866,107

(22) Filed: May 4, 2020

(30) Foreign Application Priority Data
May 8, 2019 (JP) 2019-088154

(51) Int. Cl. A61B 6/00; H04N 5/32

(52) U.S. Cl. CPC A61B 6/4476; A61B 6/58; A61B 6/566; H04N 5/32

(57) ABSTRACT

A radiography control device used in a radiography system including a radiography device and a radiography control device that controls the radiography device, the radiography control device includes: an inputter that inputs examination order information; and a hardware processor that selects guidance for guiding a dynamic state of a subject based on the order information input from the inputter.

The entire disclosure of Japanese patent Application No. 2019-088154, filed on May 8, 2019, is incorporated herein by reference in its entirety.

### BACKGROUND

### Technological Field

The present invention relates to a radiography control device (so-called console device) used in a radiography system, and the radiography system.

### Description of the Related Art

In general, a radiography system includes a radiography device provided in a radiation room and a radiography control device (so-called console device) provided in an operation room and the like that controls the radiography device.

The radiography device is provided with a radiation generation device, a radiation detection device and the like. As the radiation detection device, a flat panel detector (FPD) and the like that detects an X-ray transmitted through a subject and converts the same into image data to output is used. The FPD is provided with a convertor that converts radiation detected by a radiation detection element into an electric signal, an A/D convertor that digitizes the converted electric signal, an outputter that outputs the digitized signal to the console device as a shot image, a battery that supplies electric power to them and the like.

The radiography control device is a computer that principally controls the radiography device, displays a radiograph obtained from the FPD, indicates an image processing content and the like related to data of the radiograph and the like. The radiography control device is provided with a processor, an operation inputter such as a mouse and a keyboard, and a display such as a liquid crystal display (LCD), and controls, displays, and indicates as described above through various screens such as a menu screen displayed on the display.

Note that, in general imaging in the radiography system, kymography (serial imaging) in which still images are continuously and repeatedly shot is performed in addition to normal still image shooting. In this kymography, since it is necessary to image a breathing cycle of a patient, a breathing timing of the subject and an imaging timing are synchronized with each other by audio guidance and the like.

Since it is practically difficult for a technician to provide accurate guidance, disclosed is a technology of providing guidance that guides a dynamic state of the subject by a device such that the breathing timing of the subject and the imaging timing are synchronized with each other as disclosed in JP 2004-412 A, for example.

By the way, in radiography in recent years, the number of imaging types increases, and it is necessary for an operator to change guidance according to the imaging type. It is also necessary to change the guidance depending on a patient.

In general, the operator shoots images of dozens of subjects a day, so that there is possibility that the operator forgets to switch the guidance according to the imaging type or the patient. In that case, it is imaged while guiding the subject by erroneous guidance, so that an intended radiograph cannot be obtained. As a result, re-imaging is required, and the subject might be exposed to unnecessary radiation.

### SUMMARY

An object of the present invention is to provide a radiography control device and a radiography system capable of preventing an output of erroneous guidance due to erroneous selection of the guidance by the operator.

To achieve the abovementioned object, according to an aspect of the present invention, a radiography control device used in a radiography system including a radiography device and a radiography control device that controls the radiography device reflecting one aspect of the present invention comprises: an inputter that inputs examination order information; and a hardware processor that selects guidance for guiding a dynamic state of a subject based on the order information input from the inputter.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a connection diagram illustrating a system configuration according to an embodiment;

FIG. 2 is a block diagram illustrating a substantial configuration of a radiography control device according to the embodiment;

FIG. 3 is a view illustrating an example of order information input from an RIS to the radiography control device;

FIG. 4 is a view illustrating an example of contents of an imaging order;

FIG. 5 is a view illustrating an example of a screen displayed on the radiography control device at the time of radiography;

FIG. 6 is a view illustrating an example of guidance displayed on a display device;

FIG. 7 is a view illustrating an example of audio guidance output from a speaker;

FIG. 8 is a flowchart illustrating operation by a radiography system according to the embodiment; and

FIG. 9 is a view illustrating an example of a screen for manually adjusting volume of audio guidance.

### DETAILED DESCRIPTION OF EMBODIMENTS

Hereinafter, one or more embodiments of the present invention will be described with reference to the drawings. However, the scope of the invention is not limited to the disclosed embodiments.

### <1> SYSTEM CONFIGURATION

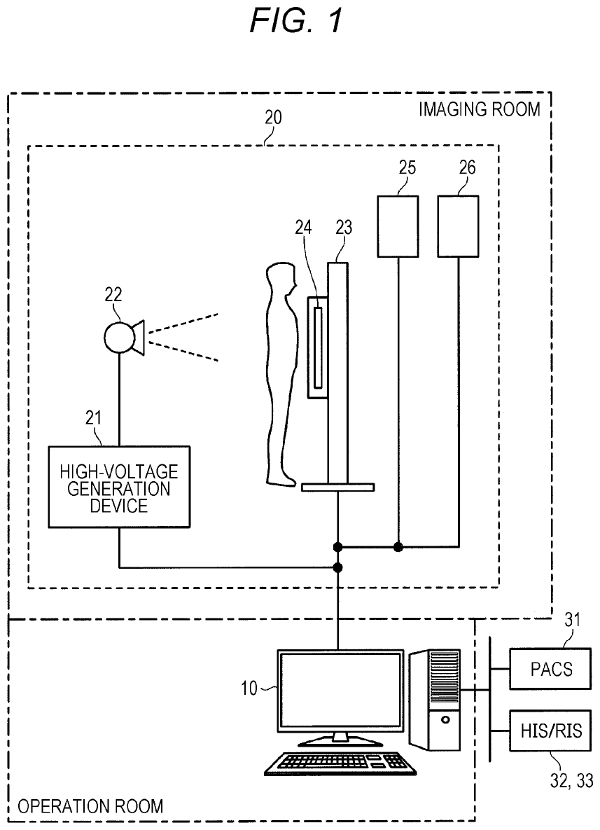

FIG. 1 is a connection diagram illustrating a system configuration of this embodiment. A radiography system is formed of a radiography device 20 provided in an imaging room and a radiography control device (console device) 10 provided in an operation room. To the radiography control device 10, picture archiving and communication systems (PACS) 31, hospital information systems (HIS) 32, and radiology information systems (RIS) 33 are connected via a communication network. In the communication network including the radiography system, the PACS 31, the HIS 32, and the RIS 33, information is transmitted and received in accordance with the digital image and communications in medicine (DICOM) standards, for example.

The radiography device 20 is provided with a high-voltage generation device 21, an X-ray tube device 22, an imaging table 23, and an FPD 24. Since well-known configurations may be adopted as the configurations, description thereof is herein omitted. In addition to such configuration, the radiography device 20 includes a display device 25 and a speaker 26. The display device 25 and the speaker 26 serve as an output device for outputting guidance for guiding a dynamic state of a subject. Therefore, in the following description, the display device 25 and the speaker 26 are sometimes referred to as the output device.

The radiography device 20 is connected to the radiography control device 10 via a communication cable. The radiography control device 10 controls imaging operation of the radiography device 20 by controlling the high-voltage generation device 21. The radiography control device 10 also performs image analysis and the like of a radiograph obtained by the FPD 24.

The radiography control device 10 inputs examination order information from the HIS 32 or the RIS 33 and displays the order information on a screen. An operator performs control operation of the radiography device 20 based on the displayed order information. The radiography control device 10 may also have a function of automatically controlling the radiography device 20 based on the order information.

### <2> CONFIGURATION OF RADIOGRAPHY CONTROL DEVICE

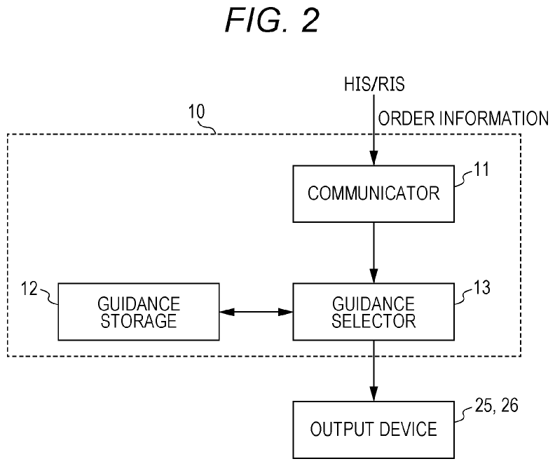

FIG. 2 is a block diagram illustrating a substantial configuration of the radiography control device 10 according to this embodiment. It goes without saying that the radiography control device 10 has an original basic configuration other than the configuration illustrated in FIG. 2.

As the basic configuration, the radiography control device 10 is provided with a controller that controls radiography in the radiography device 20, a display that displays the radiograph (for example, X-ray image) obtained from the FPD 24, and an image processor that performs image processing on such radiograph.

In addition to such basic configuration, as illustrated in FIG. 2, the radiography control device 10 is provided with a communicator 11, a guidance storage 12, and a guidance selector 13.

The communicator 11 serves as an inputter that inputs the examination order information from the HIS 32 and the RIS 33.

The guidance storage 12 stores guidance information for guiding the dynamic state of the subject in association with an imaging type, an imaging technique, patient information and the like included in the order information.

The guidance selector 13 selects guidance based on the order information. The guidance selector 13 transmits the selected guidance to the output device (display device 25 and speaker 26) in the imaging room. Note that, a process by the guidance selector 13 may be realized based on a program by a computer provided with a central processing unit (CPU), a read only memory (ROM), and a random access memory (RAM). That is, the CPU may read the program corresponding to a processing content from the ROM, expands the same on the RAM, and perform a guidance selecting process in cooperation with the expanded program.

As a result, the guidance based on the order information is output from the output device (display device 25 and speaker 26) in the imaging room.

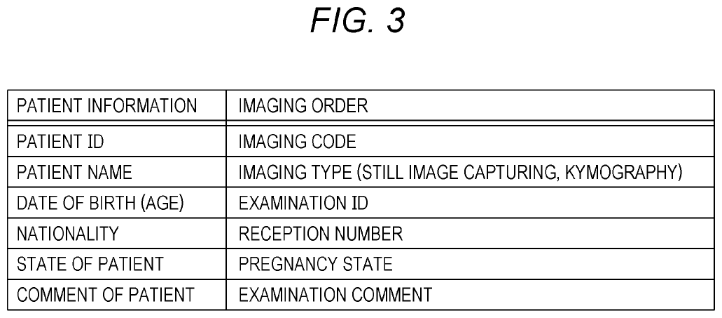

FIG. 3 illustrates an example of the order information input from the RIS 33 to the radiography control device 10. As is understood from the drawing, the order information includes the patient information and imaging order information. The patient information includes information indicating age of a patient, nationality, and a state of the patient. The imaging order includes the imaging type (still image shooting, kymography), an examination ID, and a pregnancy state.

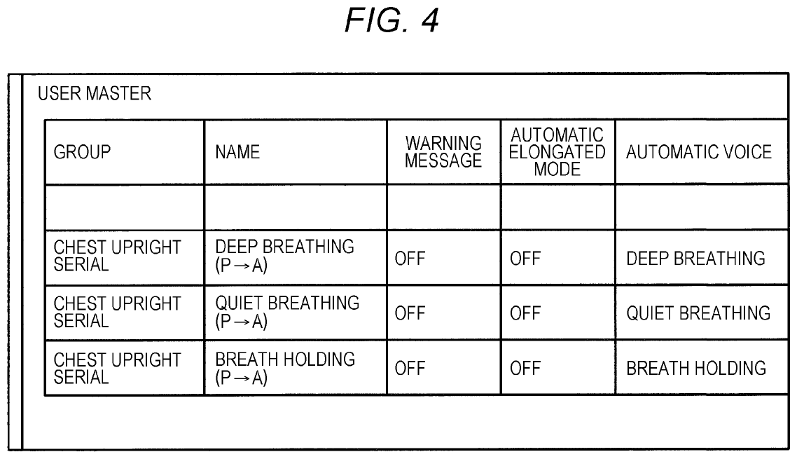

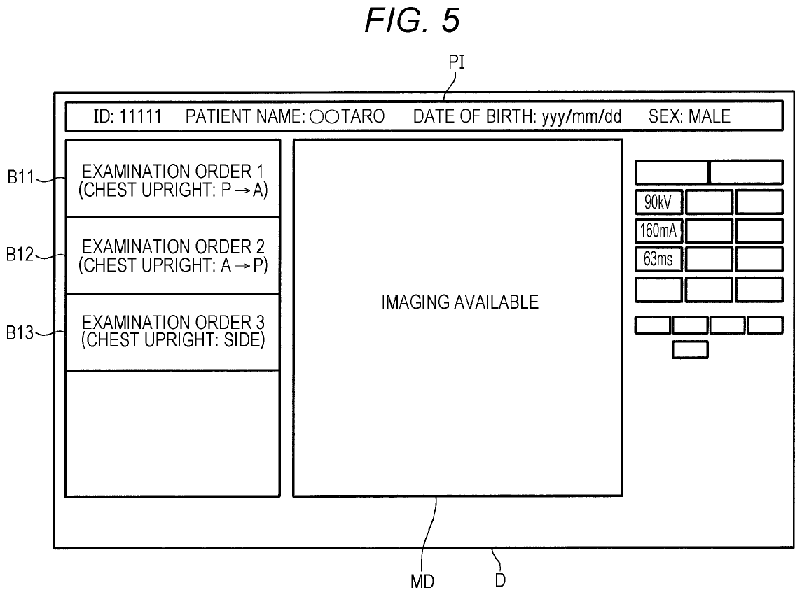

FIGS. 4 and 5 are views illustrating an example of the imaging order. FIG. 4 illustrates an example of contents of the imaging order, and FIG. 5 illustrates an example of a screen displayed on the display of the radiography control device 10 at the time of imaging.

FIGS. 4 and 5 illustrate three orders. As is understood from FIG. 4, in an order 1 (upper-stage order), the imaging type is serial (that is, kymography) and the imaging technique is deep breathing. In an order 2 (middle-stage order), the imaging type is serial and the imaging technique is quiet breathing. In an order 3 (lower-stage order), the imaging type is serial and the imaging technique is breath holding.

In FIG. 5, patient information PI is displayed on an upper side of a display screen D, and setting keys B11, B12, and B13 for indicating execution of examination orders are displayed on a left side. In addition, in a central area MD of the display screen D, an imaging condition and the like corresponding to the selected examination order is displayed. When the setting key B11 is operated on the screen in FIG. 5, the radiography control device 10 configures setting of the radiography device 20 for the order 1, and selects the guidance corresponding to the order 1 by the guidance selector 13. Similarly, when the setting key B12 is operated, the radiography control device 10 configures the setting of the radiography device 20 for the order 2, and selects the guidance corresponding to the order 2 by the guidance selector 13.

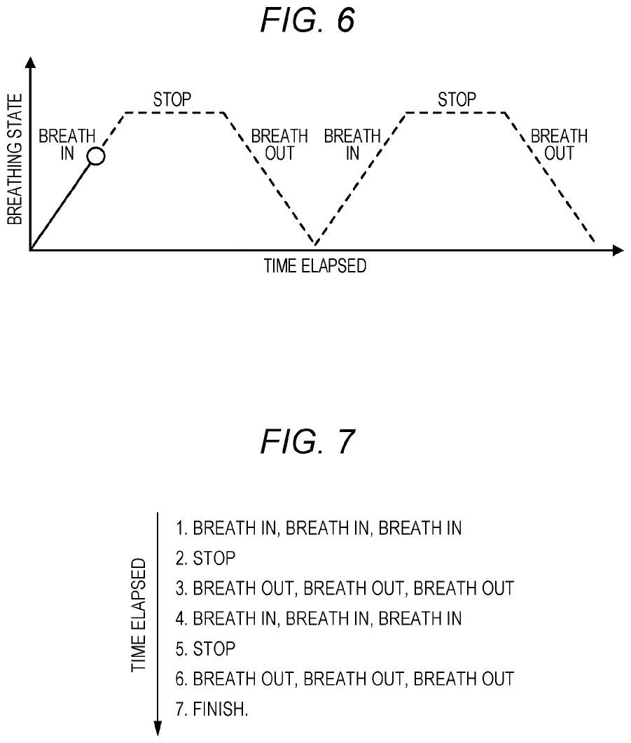

FIG. 6 illustrates an example of the guidance displayed on the display device 25. FIG. 7 illustrates an example of audio guidance output from the speaker 26. When the subject breathes according to the guidance, a breathing timing of the subject and an imaging timing are synchronized with each other, so that excellent radiography may be performed.

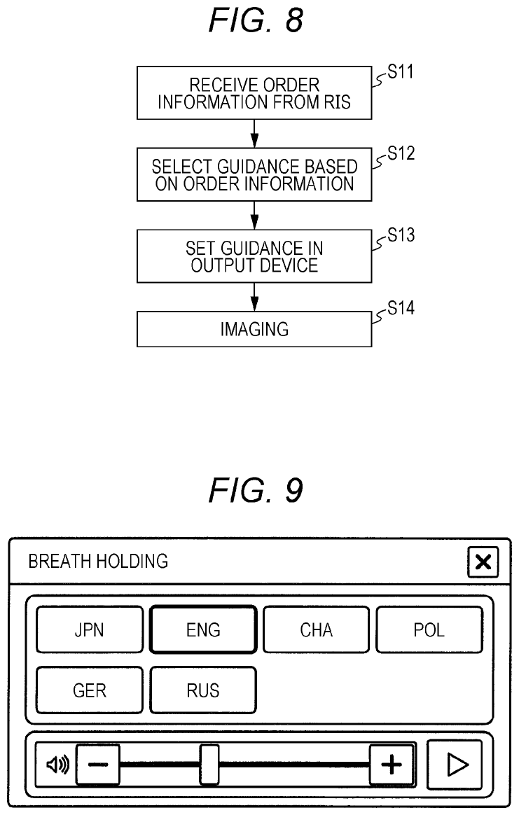

FIG. 8 is a flowchart illustrating operation by the radiography system according to this embodiment.

At step S11, the radiography control device 10 receives the order information from the RIS 33. It goes without saying that the order information may also be received from the HIS 32. At step S12, the guidance selector 13 of the radiography control device 10 selects the guidance corresponding to the order information, and sets the guidance selected at step S13 in the output device (display device 25 and speaker 26). Next, at step S14, the imaging by the radiography device 20 is performed. At the time of this imaging, the set guidance is output from the output device (display device 25 and speaker 26).

In the above-described configuration, the radiography system according to this embodiment may output the guidance suitable for the imaging type, the imaging technique, and the patient information.

For example, in a case where the imaging type of the order information is the kymography (sequence imaging), the guidance corresponding to the kymography is output, and in a case where the imaging type is the still image, the guidance corresponding to the still image shooting is output.

In addition, depending on the imaging technique of the order information which is any one of the deep breathing, the quiet breathing, or the breath holding, the guidance corresponding to the same is output.

In addition, the guidance according to the age in the patient information is output. For example, the guidance at higher audio level is output for the patient (subject) who is older than predetermined age. The guidance in a language corresponding to the nationality in the patient information is output. In addition, the guidance according to the state of the patient in the patient information may be output.

Herein, an example of guidance selection based on the order information is described, but it goes without saying that the present invention is not limited to these examples.

### <3> SUMMARY

As described above, according to this embodiment, since the radiography control device 10 is provided with the communicator 11 as the inputter that inputs the examination order information and the guidance selector 13 that selects the guidance for guiding the dynamic state of the subject based on the order information, it is possible to prevent an output of erroneous guidance due to erroneous selection of the guidance by the operator by effectively utilizing the order information.

The above-described embodiment merely describes an example of embodiment when carrying out the present invention, and the technical scope of the present invention cannot be interpreted in a limited manner by the same. That is, the present invention may be variously carried out without departing from the gist or the principal characteristics thereof.

In addition to the above-described embodiment, in a case where the patient information included in the order information indicates the same patient, the guidance of a current examination may be selected with reference to past guidance and examination result. For example, in a case where re-examination was needed because of low volume of the audio guidance in the previous examination, the guidance at higher volume than that in the previous examination is output in the current examination.

In addition to the above-described embodiment, an operator capable of manually changing the guidance may be further provided. For example, as illustrated in FIG. 9, a screen for manually adjusting the volume of the audio guidance is displayed so that the operator may adjust the volume while watching the state of the subject.

In addition to the above-described embodiment, different audio guidance may be selected to be output to the imaging room in which the radiography device 20 is arranged and the control room in which the radiography control device 10 is arranged. For example, the audio guidance at volume corresponding to the order information may be output to the imaging room, whereas the audio guidance indicating a remaining time of the examination based on the order information may be output to the operation room.

Although the guidance for guiding the breathing timing is described as an example in the above-described embodiment, the guidance of the present invention is not limited to the breathing timing, and may be, for example, a body position.

In the above-described embodiment, a case is described in which the guidance selector 13 is provided on the radiography control device 10, but the guidance selector 13 may also be provided on the radiography device 20. The guidance storage 12 may also be provided, for example, on the radiography device 20. Alternatively, the guidance information associated with the order information may be stored on the cloud, and the guidance information selected based on the order information may be read from the cloud.

The present invention is suitable for the radiography system which guides the dynamic state of the subject by the guidance.

Although embodiments of the present invention have been described and illustrated in detail, the disclosed embodiments are made for purposes of illustration and example only and not limitation. The scope of the present invention should be interpreted by terms of the appended claims

What is claimed is:

1. A radiography control device used in a radiography system including a radiography device and a radiography control device that controls the radiography device, the radiography control device comprising:
an inputter that inputs examination order information; and
a hardware processor that selects guidance for guiding a dynamic state of a subject based on the order information input from the inputter.

2. The radiography control device according to claim 1,
wherein the order information is output from an HIS server or an RIS server.

3. The radiography control device according to claim 1,
wherein the order information includes an imaging type, an imaging technique, and/or patient information, and
the hardware processor selects the guidance based on the imaging type, the imaging technique, and/or the patient information.

4. The radiography control device according to claim 3, further comprising
a storage in which a plurality of pieces of guidance is stored in association with the imaging type, the imaging technique, and/or the patient information,
wherein the hardware processor selects guidance corresponding to the input order information from the plurality of pieces of guidance in the storage.

5. The radiography control device according to claim 1,
wherein the hardware processor selects the guidance with reference to past guidance and an examination result in a case where patient information included in the order information indicates the same patient.

6. The radiography control device according to claim 1,
wherein the hardware processor selects guidance at a voice level according to patient information included in the order information.

7. The radiography control device according to claim 1, further comprising
an operator capable of manually changing the guidance.

8. The radiography control device according to claim 1,
wherein the hardware processor selects different audio guidance for an imaging room in which the radiography device is arranged, and a control room in which the radiography control device is arranged.

9. A radiography system including a radiography device and a radiography control device that controls the radiography device, the radiography system comprising:
an inputter that inputs order information;
a hardware processor that selects guidance for guiding a dynamic state of a subject based on the order information; and
a guidance outputter that outputs the guidance selected by the hardware processor so as to be perceptible to a subject.

10. The radiography system according to claim 9,
wherein the order information is output from an HIS server or an RIS server.

11. The radiography system according to claim 9,
wherein the inputter and the hardware processor are provided on the radiography control device, and
the guidance outputter is provided at least in an imaging room in which the radiography device is arranged.

12. The radiography system according to claim 9,
wherein the order information includes an imaging type, an imaging technique, and/or patient information, and
the hardware processor selects the guidance based on the imaging type, the imaging technique, and/or the patient information.
